(19) United States
(12) Patent Application Publication
(10) Pub. No.: US 2008/0171991 A1
(43) Pub. Date: Jul. 17, 2008

(54) Sheath tube catheter

(76) Inventor: Stephen Michael Kourakis, Sao Paulo (BR)

Correspondence Address: RYAN KROMHOLZ & MANION, S.C., POST OFFICE BOX 26618, MILWAUKEE, WI, 53226, US

(21) Appl. No.: 11/588,080

(22) Filed: Oct. 26, 2006

(51) Int. Cl. A61M 5/32

(52) U.S. Cl. 604/175

(57) ABSTRACT

The catheter comprises an internal tube that folds over and back on itself, so that the catheter rolls in, advancing at the tip. The catheter is made of a flexible material such as surgical type rubber tubing, or plastic film, etc. The plastic may intentionally be rougher on the external surface than on the internal walls.

### BACKGROUND OF THE INVENTION

A catheter is a tube that can be inserted into a body cavity duct or vessel, allowing drainage or injection of fluids or access by surgical instruments to target areas of a body. Catheters can be thin flexible tube, known as soft catheters, or can be hard rigid tube known as hard catheters. Traditional catheters typically consist of stiff or semi-flexible hollow tubes that are inserted into the patient.

Placement of a catheter into a particular part of the body allows procedures such as draining urine from the bladder in a urinary catheterization. Other uses include drainage of fluid collections (e.g. in an abdominal abscess); administration of intravenous fluids, medication or parenteral nutrition; angioplasty; angiography; balloon septostomy; balloon sinuplasty; direct measurement of blood pressure in an artery or vein; direct measurement of intracranial pressure; and administration of anaesthetic medication into the epidural space, the subarachnoid space, or around a major nerve bundle such as the brachial plexus.

The insertion of urinary catheters can cause discomfort and tissue trauma and may increase in the incidence of infection. Traditional catheters move against the body of the patient, an action that can dislodge bacteria and virus from distal locations and push them into the body. Also, the inward movement of a traditional catheter relative to the body may cause surgical complications at contact surfaces.

### SUMMARY OF THE INVENTION

One aspect of the present invention is the elimination of movement of the catheter against the body of the patient. This catheter requires no inward movement of the catheter wall, relative to the body, at all contact surfaces. The catheter can also be used to pull material distally, away from or out of the patient.

The catheter of the present invention comprises an internal tube that folds over and back on itself, so that it rolls in to the desired area, advancing at from the tip only. The catheter is made of a flexible material such as surgical type rubber tubing, or plastic film, etc. The plastic may intentionally be rougher on the external surface than on the internal walls.

Whereas conventional catheters slide over the surface of the patient's body and may move material along with them, the presently invented catheter does not move relative to its contact points and exerts no force to dislodge or carry material into the body. It need not slide against the patient's body upon insertion or removal.

The present invention has three embodiments, a primary form, an expanding lumen form, and a sheathed embodiment, described below.

### BRIEF DESCRIPTION OF THE DRAWINGS

FIG. 1a shows a perspective view of a primary catheter form of the present invention, with the tube folded back upon itself;

FIG. 1b shows a perspective view of the primary catheter form of the present invention, with the tube folded back upon itself;

FIG. 1c shows a perspective view of the primary catheter form of the present invention, with the tube folded back upon itself;

FIG. 1d shows a cross-sectional view of the primary catheter form of the present invention at two stages of insertion;

FIG. 2a shows a perspective view of an expanding-lumen embodiment of the present invention;

FIG. 2b shows a perspective view of the expanding-lumen embodiment of the present invention through various stages of expansion and contraction;

FIG. 3a shows a cross-sectional view of a sheathed catheter form of the present invention;

FIG. 3b shows an exploded view of the sheathed catheter form of the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention that may be embodied in other specific structures. While the preferred embodiment has been described, the details may be changed without departing from the invention, which is defined by the claims.

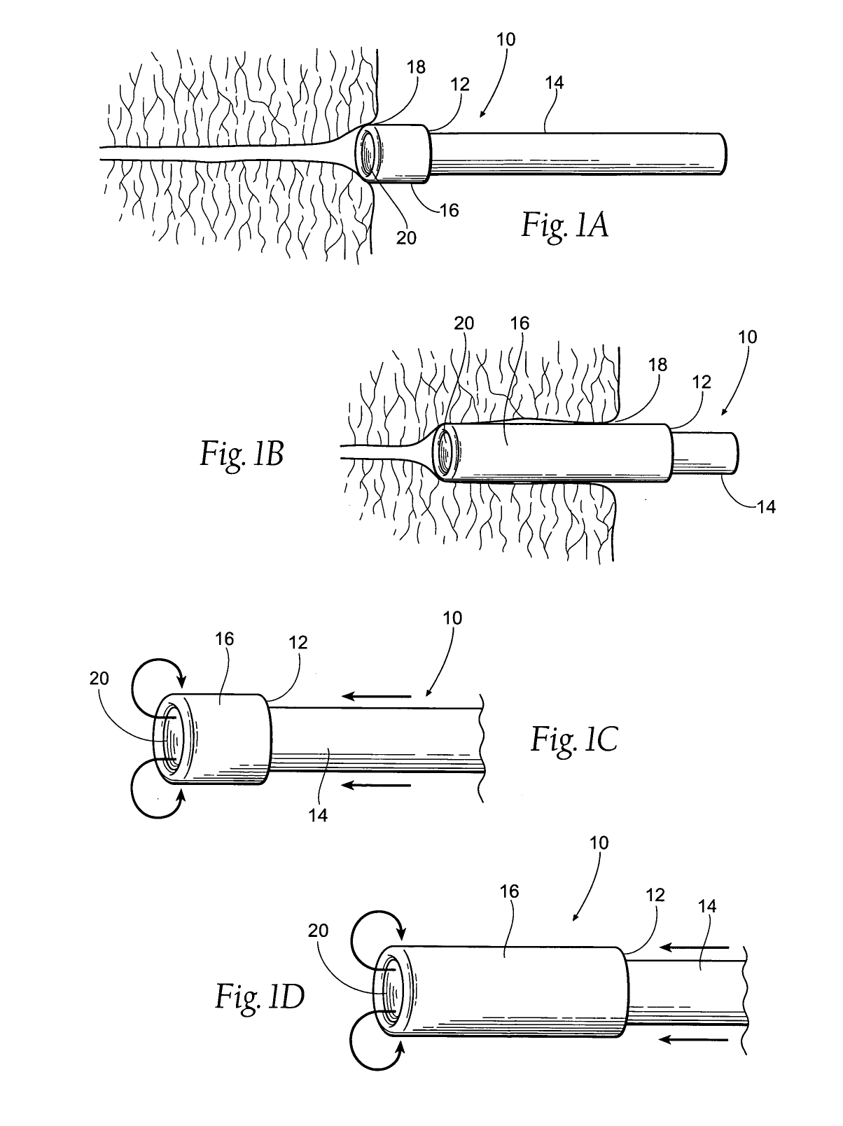

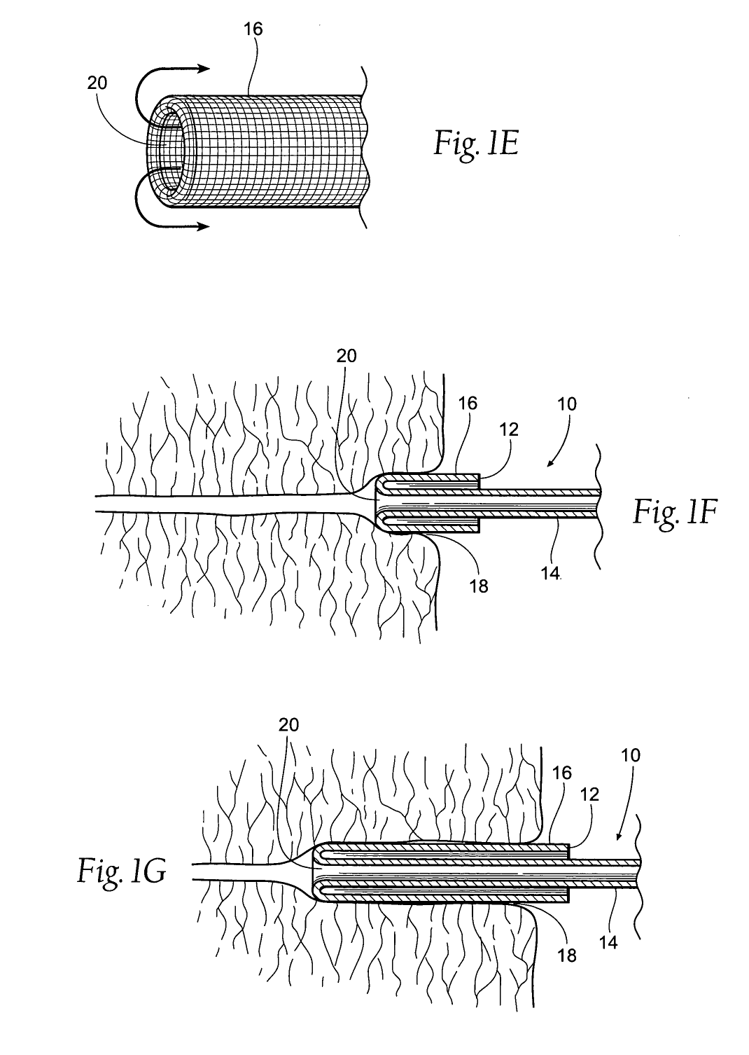

Referring now to FIG. 1a, a perspective view of a primary catheter form 10 of the present invention is shown, with the catheter tube 10 folded back upon itself. A distal end 12 of the tube 10 is folded back upon itself so that the exterior 14 of the tube 10 is covered by the interior 16 of the tube 10 as the leading edge 20 of the tube is inserted into body passage 18.

By pushing flexible tube 10 into the body passage 18, the leading edge 20 advances from the inside, and the leading edge 20 moves forward. More of the interior 16 of the tube 10 is pushed back over exterior 14 of the tube 10 during advancement. In this manner, leading edge 20 proceeds to the catheterization target, in accordance with the medical objective.

FIGS. 1b and 1d (a cross-sectional view), show that by pushing the tube 10 into body passage 18 (shown in FIG. 1a), tube 10 and particularly leading edge 20 crawls forward, with the exterior 14 exposing the interior 16, while distal end 12 remains relatively stationary.

The catheter 10 may be removed by pulling back on the exterior 14 such that catheter 10 rolls out just as it went in. Alternatively, by pulling distal end 12 along simultaneously with exterior 14, the catheter 10 slides against the patient's tissue and may be used in a scrape like fashion to clean the insertion tract, pulling bacteria or other substances distally, away from the patient.

FIG. 1c shows a closeup of the leading edge 20, exposing exterior 16 during advancement.

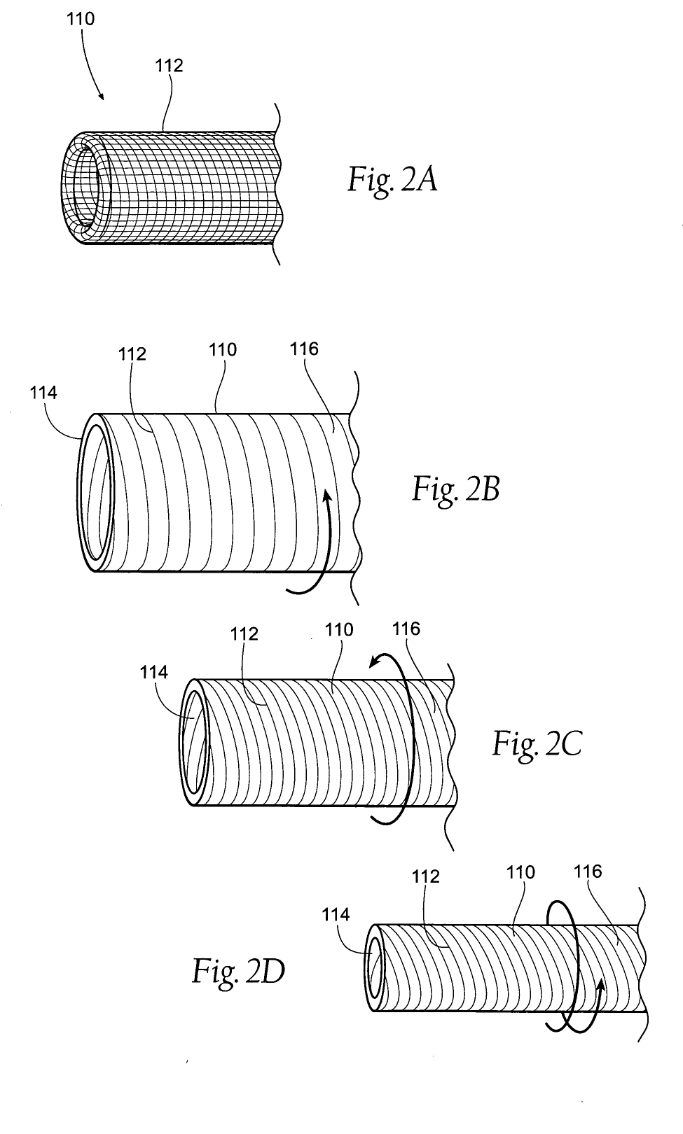

FIGS. 2a and 2b illustrate a perspective view of an expanding-lumen flexible catheter 110. In this embodiment, a preferably spiral weakness zone 12 is integrated with the tube 10. As can be seen, the zone of weakness trends in either a counterclockwise-upward fashion (shown), or a counterclockwise-downward fashion (not shown), such that rotation of a first end 116 of the tube 110 in a counterclockwise direction relative to the second end of the tube 114 causes a radial expansion of tube 110. Alternatively, rotation of a first end 116 of the tube 110 in a clockwise direction relative to the second end of the tube 114 causes a radial contraction of tube 110.

Note that the zone of weakness 112 can longitudinally extend equal to or less than the longitudinal length of tube 110, such that portions of tube 110 can be expanded or contracted radially.

This expansion/contraction in the radial direction allows the catheter to be smaller on insertion (and on removal if desired), and to open or close to control drainage. The expansion of the diameter of the catheter can also be used to increase friction against the patient's body when performing a cleaning removal as mentioned above.

Inherent in both embodiments of the tube 10 and 110, is the possibility for the catheter to be applied in cleaning procedures. For instance, the tubes and 10 and 110 (which may or may not have the purpose of fluid drainage also) rolls in on insertion, and is pulled out on removal. On insertion no sliding of the catheter against the patient exists to push bacteria or other substances farther into the patient, whereas upon removal there is a sliding of the catheter against the body that will pull matter distally, out of the body.

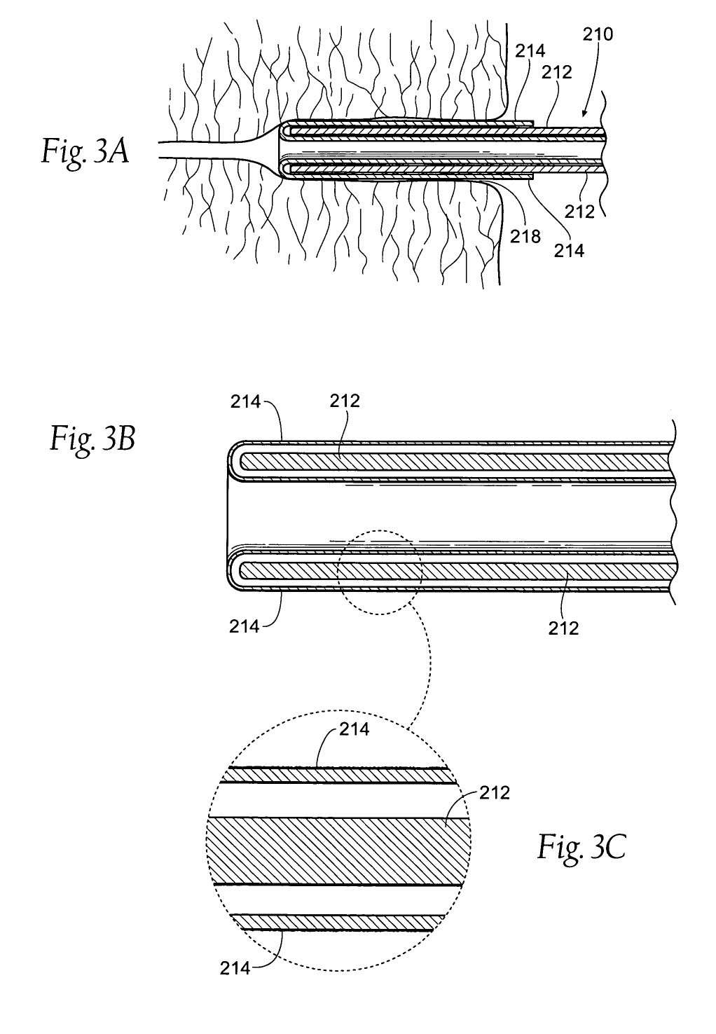

Referring now to FIG. 3a, a cross-sectional view of a sheathed catheter form 210 of the present invention is shown. In this embodiment, a hollow tube 212 with a flexible film 214 that passes up through its center and folds down over its outer surface is provided. On insertion, the film 214 slides through the hollow tube 212 in roll-like fashion as described above. The Tube advances into the patient through passage 18, and the outer film 18 slides against the tube 212, but does not move relative to the patient. FIG. 3b, shows an exploded view of the sheathed catheter 210. As can be seen in the exploded view of FIG. 3b, the tube 10, 110 or 210 can be provided with texturing.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A flexible catheter comprising:
a flexible tube comprising a distal end, an interior and an exterior;
said distal end folded over forming a leading edge;
said distal end exposing said interior of said flexible tube and covering the exterior of said flexible tube;
said distal end positioned outside a body passage;
said exterior of said tubing manipulated to advance said leading edge interiorly into said body passage while said distal end remains proximal to said outside of said body passage;
said leading edge presenting a catheterization site interior to said body.

2. A flexible catheter according to claim 1, said catheter further comprising a rigid tube having a first end and a second end;
one of said first and second ends positioned between said interior and said exterior of said tube proximal to said leading edge.

3. A flexible catheter according to claim 1, said tube comprising a zone of weakness that expands or contracts said tube in response to rotation of one end of said tube.
